(19) United States
(12) Patent Application Publication
(10) Pub. No.: US 2011/0008395 A1
(43) Pub. Date: Jan. 13, 2011

(54) MIXED MICELLES INCLUDING AMPHIPATHIC CONJUGATES OF RNA AGENTS, AND USES THEREOF

(76) Inventors: Vladimir Torchilin, Charlestown, MA (US); Tatyana S. Levchenko, Revere, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/667,713

(22) PCT Filed: Jul. 7, 2008
(86) PCT No.: PCT/US08/08326

§ 371 Date: Jan. 5, 2010

Related U.S. Application Data
(60) Provisional application No. 60/948,433, filed on Jul. 6, 2007.

(51) Int. Cl. A61K 31/713; A61K 9/00; A61P 35/00; A61P 37/00; A61P 5/00; A61P 7/00

(52) U.S. Cl. 424/400; 514/44 A

(57) ABSTRACT

Disclosed are improved pharmaceutical formulations for the delivery of RNA interference agents, such as antisense RNA, micro-RNA and siRNA. The formulations employ mixed micelles including amphipathic conjugates of the iRNA agents and amphipathic micelle-forming molecules with extended hydrophilic chains. Also disclosed are methods of using the pharmaceutical formulations to increase delivery of an iRNA agent to an intracellular target, and to decrease extracellular nuclease degradation of an iRNA agent in the formulations.

### RELATED APPLICATION

This application claims benefit of priority to U.S. Provisional Application No. 60/948,433, filed Jul. 6, 2007.

### FIELD OF THE INVENTION

The invention relates to the field of pharmaceutical sciences, and particularly formulations employing mixed micelles, and specifically the formulation of mixed micelles including amphipathic conjugates of RNA agents, such as siRNAs, and uses thereof.

### BACKGROUND OF THE INVENTION

Many diseases (e.g., cancers, hematopoietic disorders, endocrine disorders, and immune disorders) arise from the abnormal expression or activity of a particular gene or group of genes. Similarly, disease can result through expression of a mutant form of protein, as well as from expression of viral genes that have been integrated into the genome of their host. The therapeutic benefits of being able to selectively silence these abnormal or foreign genes are obvious.

Oligonucleotide compounds have important therapeutic applications in medicine. Oligonucleotides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. Antisense RNA, micro-RNA and small interfering RNA (siRNA) are different types of oligonucleotides that prevent the formation of corresponding proteins by gene-silencing.

Antisense methodology is the complementary hybridization of relatively short oligonucleotides (e.g., 13-30 nucleotides) to mRNA or DNA such that the normal, essential functions, such as protein synthesis, of these intracellular nucleic acids are disrupted. The hybridization is by sequence-specific hydrogen bonding via Watson-Crick base pairs of oligonucleotides to RNA or single-stranded DNA, and interferes with the transcription and/or translation of the target nucleic acid sequence.

Micro-RNAs are a large group of small RNAs produced naturally in organisms, at least some of which regulate the expression of target genes. Micro-RNAs are formed from an approximately 70 nucleotide single-stranded hairpin precursor transcript by the ribonuclease Dicer (Ambros et al. (2003), Current Biology 13(10):807-818), which cleaves the precursor to form 21-23 nucleotide double-stranded micro-RNAs. In many instances, the micro-RNA is transcribed from a portion of the DNA sequence that previously had no known function. Micro-RNAs are not translated into proteins but, rather, they bind to specific messenger RNAs blocking translation. It is thought that micro-RNAs base-pair imprecisely with their targets to inhibit translation.

RNA interference or “RNAi” is a term initially coined by Fire and co-workers to describe the observation that double-stranded RNA (dsRNA) can block gene expression when it is introduced into worms (Fire et al. (1998), Nature 391:806-811). Short dsRNA directs gene-specific, post-transcriptional silencing in many organisms, including vertebrates, and has provided a new tool for studying gene function. RNAi is mediated by an RNA-induced silencing complex (RISC), a sequence-specific, multicomponent nuclease that destroys messenger RNAs homologous to the silencing trigger.

Treatment with dsRNA has become an important method for analyzing gene functions in invertebrate organisms. For example, Dzitoveva et al. showed that RNAi can be induced in adult fruit flies by injecting dsRNA into the abdomen of anesthetized Drosophila, and that this method can also target genes expressed in the central nervous system (Dzitoveva et al. (2001), Mol. Psychiatry 6(6):665-670). Both transgenes and endogenous genes were successfully silenced in adult Drosophila by intra-abdominal injection of their respective dsRNA. Moreover, Elbashir et al. provided evidence that the direction of dsRNA processing determines whether sense or antisense target RNA can be cleaved by an siRNA-protein complex (Elbashir et al. (2001), Genes Dev. 15(2):188-200).

Since the first report of the phenomenon of RNA interference (RNAi) in 1998 (Fire et al. (1998), Nature 391:806-811), there has been a wave of interest in the development of diagnostic and therapeutic strategies based on the use of synthetic siRNAs, since those are clearly seen as a potential new class of pharmaceutical drugs (Bumcrot et al. (2006), Nat. Chem. Biol. 2:711-719). Several RNAi-based in vivo strategies have been reported in the literature for the treatment of a wide range of conditions from viral infections (Giladi et al. (2003), Mol. Ther. 8:769-776; Song et al. (2003), Nat. Med. 9:347-351) to cancer (Duxbury et al. (2003), Biochem. Biophys. Res. Commun. 311:786-792; Duxbury et al. (2004), Oncogene 23:1448-1456) and even neurological conditions (Dorn et al. (2004), Nucleic Acids Res. 32:e49; Thakker et al. (2005), Mol. Psychiatry 10:782-789, 714).

Naked siRNA has been administered intravenously (Zender et al. (2003), Proc. Natl. Acad. Sci. USA 100:7797-7802), intrathecally (Dorn et al. (2004), Nucleic Acids Res. 32:e49), intraperitoneally (Filleur et al. (2003), Cancer Res. 63:3919-3922) and, where applicable, by direct injection into the target tissue (Zender et al. (2003), Proc. Natl. Acad. Sci. USA 100:7797-7802; Aharinejad et al. (2004), Cancer Res. 64:5378-5384; Lingor et al. (2005), Brain 128:550-558; Pille et al. (2005), Mol. Ther. 11:267-274). Appreciable levels of activity, especially in liver tissue in the case of hydrodynamic IV injection, have been reported (Giladi et al. (2003), Mol. Ther. 8:769-776; Song et al. (2003), Nat. Med. 9:347-351; Zender et al. (2003), Proc. Natl. Acad. Sci. USA 100:7797-7802; Bradley et al. (2005), Pancreas 31:373-379; Hamar et al. (2004), Proc. Natl. Acad. Sci. USA 101:14883-14888; Hino et al. (2006), Biochem. Biophys. Res. Commun. 340:263-267; Tompkins et al. (2004), Proc. Natl. Acad. Sci. USA 101:8682-8686). A general progress towards in vivo use of siRNAs has been recently reviewed in (Behlke (2006), Mol. Ther. 13:644-670), and new RNAi-based therapeutics are actively developing (Liu et al. (2007), Histol. Histopathol. 22:211-217). Unfortunately, the applicability of many of the alternative routes mentioned is limited to only a few tissues and, in the case of direct injection, further limited to easily accessible tissues (Aigner (2006), J. Biomed. Biotechnol. 2006(4):71659). Further, high doses and repeated administration are often necessary for activity.

While naked siRNA approaches continue to be pursued for clinical application, there is now a growing acceptance that a major hurdle now facing the application of siRNA based strategies to practical clinical therapy is the need for efficient delivery of siRNA to the site of intended action (Aigner (2006), J. Biomed. Biotechnol. 2006(4):71659). Although chemical modification of siRNA molecules (including their modification with cholesteryl residues) to achieve better stability and confer tissue-specific targeting has attracted some attention (Soutschek et al. (2004), Nature 432:173-178; Manoharan (2004), Curr. Opin. Chem. Biol. 8:570-579; Morrissey et al. (2005), Nat. Biotechnol. 23:1002-1007), the major focus has been on the use of carrier systems to improve the delivery of siRNA. As was noted by Bumcort et al. “Effective delivery is the most challenging remaining consideration for successful translation of RNAi to the clinic” (Bumcort et al. (2006), Nat. Chem. Biol. 2:711).

Approaches towards improved siRNA delivery have understandably borrowed much from the already established field of DNA delivery, with both viral and non-viral based strategies being explored. The drawbacks of viral approaches already identified from DNA based therapy, including immune responses and possible oncogenesis, are potentially more limiting in the delivery of siRNA because of the short duration of action and the need for repeated administration (Bartlett et al. (2006). Nucleic Acids Res. 34:322-333). Non-viral systems are comparatively more flexible in design and are easier to formulate. The potential for repeated administration is greater due to improved biocompatibility, and non-viral systems can be used to deliver both DNA coding for siRNA products or the siRNA duplexes themselves.

Polymer-based siRNA delivery systems, particularly polyethyleneimine (PEI)-based ones, have been actively investigated (Aigner (2006), J. Biomed. Biotechnol. 2006(4):71659; Putnam et al. (2006), Crit. Rev. Ther. Drug Carrier Syst. 23:137-164), and cationic polymers, particularly PEI, have been used with some success to improve the efficacy of siRNA activity in vivo (Ge et al. (2004), Proc. Natl. Acad. Sci. USA 101:8676-8681; Leng et al. (2005), Cancer Gene Ther. 12:682-690; Urban-Klein et al. (2005), Gene Ther. 12:461-466; Yin et al. (2005), J. Mol. Cell. Cardiol. 39:681-689), although the toxicity of such carriers remains an issue. Polymeric nanoparticles have also been considered as potential siRNA carriers (Toub et al. (2006), Pharmacother. 60:607-620). Nanoparticle carrier systems based on polymer or metal nanoparticles are also being explored for siRNA delivery (Schiffelers et al. (2004), Nucleic Acids Res. 32:e149; Derfus et al. (2007), Bioconjug. Chem. 18(5):1391-6).

Lipidic carriers of siRNA have naturally drawn a lot of attention (Spagnou et al. (2004), Biochemistry (Mosc). 43:13348-13356). Liposomes appear to be the most widely explored of the non-viral systems for siRNA therapy (Aigner (2006), J. Biomed. Biotechnol. 2006(4):71659). Liposome formulations of siRNA have been used to improve specific gene knock-down in the treatment of solid tumors (Landen et al. (2005), Cancer Res. 65:6910-6918), metastatic cancer (Yano et al. (2004), Clin. Cancer Res. 10:7721-7726) and a variety of other conditions in animal models (Nogawa et al. (2005), J. Clin. Invest. 115:978-985). Our own experience with the liposome-based siRNA delivery systems involving cell-penetrating peptide-mediated intracellular delivery (Zhang et al. (2006), J. Control. Release 112:229-239), although resulting in some success, was associated with reproducibility problems as well as with issues relating to preparation and scalability. First attempts to prepare ligand-targeted nanocarrier-based siRNA delivery systems have also been described (Ikeda et al. (2006), Pharm. Res. 23:1631-1640). However, none of the strategies tested to date has resulted in clinically significant systems.

siRNA has been shown to be extremely effective as a potential anti-viral therapeutic with numerous published examples appearing recently. siRNA molecules directed against targets in the viral genome dramatically reduce viral titers by orders of magnitude in animal models of influenza (Ge et al. (2004), Proc. Natl. Acad. Sci. USA 101:8676-8681; Tompkins et al. (2004), Proc. Natl. Acad. Sci. USA 101:8682-8686; Thomas et al. (2005), Expert Opin. Biol. Ther. 5:495-505 (2005)), respiratory syncytial virus (RSV) (Bitko et al. (2005), Nat. Med. 11:50-55), hepatitis B virus (HBV) (Morrissey et al. (2005), Nat. Biotechnol. 23:1002-1007), hepatitis C virus (Kapadia (2003), Proc. Natl. Acad. Sci. USA 100:2014-2018; Wilson et al. (2003), Proc. Natl. Acad. Sci. USA 100:2783-2788) and SARS coronavirus (Li et al. (2005), Nat. Med. 11:944-951). In addition, research is currently underway to develop interference RNA therapeutic agents for the treatment of many diseases including central-nervous-system diseases, inflammatory diseases, metabolic disorders, oncology, infectious diseases, and ocular disease.

Despite advances in antisense RNA, micro-RNA and siRNA, technologies, one of the major hurdles is the intracellular delivery of these oligonucleotides into the cells. In particular, there is substantial nuclease activity in both the extracellular and intracellular environments which rapidly degrades RNA administered in vivo. As a result, only a very small proportion of the RNA which is administered to a subject reaches the desired target in cells. Although a wide variety of delivery strategies have been investigated in the art (see, e.g., Gilmore et al. (2006), Current Drug Delivery 3:147-145), many important issues for making active and clinically acceptable preparations of antisense RNA, micro-RNA and siRNA remain unresolved, and there remains a need for a simple, universal, and easily made delivery system for RNA-based therapeutic agents.

### SUMMARY OF THE INVENTION

The present invention depends, in part, upon the recognition that certain mixed micelles can be produced, which include both amphipathic conjugates of RNA moieties and micelle-forming amphipathic molecules having radially extending hydrophilic moieties, such that the radially extending hydrophilic moieties form a layer which surrounds or masks at least a portion of the RNA moieties, and thereby increases the in vivo stability of siRNA by sterically hindering and decreasing the digestion of the RNA moieties by nucleases. In some embodiments, the invention provides mixed micelles in which a hydrophilic layer has sufficient thickness and density to substantially surround or mask the RNA moieties and thereby substantially protect them from digestion by nucleases. As a result, the invention provides improved methods for delivering RNA to therapeutic targets in cells, with substantially decreased digestion by extracellular nucleases, and substantially increased RNA half-life in the blood, cerebrospinal fluid or local extracellular microenvironment. In addition, the surface of such mixed micelles can be modified with moieties that target the mRNA-containing micelles to desired target cells and/or provide enhanced intracellular penetration.

Thus, in one aspect, the invention provides pharmaceutical formulations of an iRNA agent including (1) a mixed micelle including (a) a plurality of amphipathic iRNA conjugates, each of which includes a hydrophilic moiety and a hydrophobic moiety, and each of which hydrophilic moieties includes an iRNA moiety of the iRNA agent; (b) a plurality of micelle-forming amphipathic molecules, each of which includes a hydrophilic moiety and a hydrophobic moiety, and each of which hydrophilic moieties includes at least one hydrophilic chain which extends radially from the center of the mixed micelle; and (2) a pharmaceutically acceptable carrier.

In some embodiments of the pharmaceutical formulation, the plurality of hydrophilic chains forms a hydrophilic layer extending radially from the center of said mixed micelle; and the amphipathic iRNA conjugates are positioned in the mixed micelle such that at least a portion of the surface of the iRNA moiety is within the hydrophilic layer.

In some embodiments of the pharmaceutical formulation, no portion of the surface of the iRNA moiety extends radially outward beyond the hydrophilic layer.

In some embodiments of the pharmaceutical formulation, the plurality of hydrophilic chains have an average backbone length between 200 and 500 Å. In other embodiments, the plurality of hydrophilic chains have an average backbone length between 100-1,000 Å, and between 50-2,000 Å.

In some embodiments of the pharmaceutical formulation, the plurality of hydrophilic chains have an average molecular weight between 1,500 and 5,000 g/mole. In other embodiments, the plurality of hydrophilic chains have an average molecular weight between 500 and 15,000, and between 1,000-10,000 g/mole.

In some embodiments of any of the foregoing, the hydrophobic moieties of the micelle-forming amphipathic molecules are selected from radicals of a long-chain fatty acid, a phospholipid, a lipid, and a glycolipid.

In some embodiments of any of the foregoing, the hydrophobic moieties of the amphipathic iRNA conjugates are selected from radicals of a cholesterol, a long chain fatty acid, a lipid, a phospholipid, and a glycolipid.

In some of the foregoing embodiments, the fatty acid is selected from butanoic acid, hexanoic acid, octanoic acid, decanoic acid, dodecanoic acid, tetradecanoic acid, hexadecanoic acid, octadecanoic acid, eicosanoic acid, docosanoic acid, tetracosanoic acid and unsaturated congeners thereof.

In some of the foregoing embodiments, the phospholipid is selected from phosphatidyl ethanolamine (PE), phosphatidyl choline (PC), phosphatidyl glycerol (PG), phosphatidyl inositol (PI), phosphatidyl serine (PS), phosphatidic acid (PA), and sphingomyelin.

In some of the foregoing embodiments, the glycolipid is selected from a galactolipid, a sulfolipid, a cerebroside, and a ganglioside.

In some of embodiments of any of the foregoing, the hydrophilic moieties of the micelle-forming amphipathic molecules are selected from radicals of a PEG, a PEI, a polyvinylpyrrolidone, a polyacrylamide, a polyvinyl alcohol, a polyoxazolines, a polymorpholines, a chitosan, and a water-soluble peptide.

In some of the foregoing embodiments, the hydrophilic moieties are PEG radicals having an average molecular weight between 1,500 and 5,000 g/mole. In other embodiments, the PEG radicals have an average molecular weight between 500 and 15,000, and between 1,000-10,000 g/mole.

In another aspect, the invention provides methods of increasing the delivery of an iRNA agent to an intracellular target by formulating the iRNA agent in one of the pharmaceutical formulations described herein, and administering the pharmaceutical formulation extracellularly, such that delivery of the iRNA agent to the intracellular target is increased relative to delivery of the iRNA agent in the pharmaceutically acceptable carrier.

In another aspect, the invention provides methods of decreasing extracellular nuclease degradation of an iRNA agent by formulating the iRNA agent in one of the pharmaceutical formulations described herein, and administering the pharmaceutical formulation extracellularly, such that extracellular nuclease degradation of the iRNA agent in the pharmaceutical formulation is decreased relative to degradation of the iRNA agent in the pharmaceutically acceptable carrier.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 is a graph showing the size distribution of siRNA-Chol/PEG-PE micelles made in accordance with the invention.

FIG. 2 is epifluorescence micrographs of MCF7 (human breast adenocarcinoma) cells stained with Hoechst33342 nuclear stain. FIGS. 2A and 2B: Cells exposed to Cy3-siRNA-Chol/PEG2-PE micelles for 2 hours. FIGS. 2C and 2D: Untreated cells. FIGS. 2A and 2C: UV Channel. FIGS. 2B and 2D: Red channel.

FIG. 3 is a graph showing cell-associated Cy3 fluorescence measured at Ex554, Em570 with MCF7 cells (A) and 4T1 cells (B) after the incubation for 2 h with Cy3-siRNA-Chol/PEG-PE micelles; n=3.

FIG. 4 is a graph showing decrease of GFP production after incubation of C166-GFP mouse endothelial cells with Col-SiRNA and Chol-SiRNA-PEG-PE micelles (same siRNA quantity).

FIG. 5 illustrates schemes for siRNA-Cholesterol synthesis. FIG. 5A: preparation of mercaptyl-cholesterol. FIG. 5B: conjugation of mercaptyl cholesterol and siRNA via SPDP.

FIG. 6 illustrates schemes for Cy3-siRNA-Cholesterol synthesis.

### DETAILED DESCRIPTION

### 1. Introduction

The present invention depends, in part, upon the recognition that certain mixed micelles can be produced, including amphipathic conjugates of RNAs and micelle-forming amphipathic molecules having radially extending hydrophilic moieties, such that the radially extending hydrophilic moieties form a layer which surrounds or masks at least a portion of the RNA moieties and thereby decreases digestion of the RNA moieties by nucleases. In some embodiments, the invention provides mixed micelles in which a hydrophilic layer has sufficient thickness and density to substantially surround or mask the RNA moieties and thereby substantially protect them from digestion by nucleases. As a result, the invention provides improved methods for delivering RNA to therapeutic targets in cells, with substantially decreased digestion by extracellular nucleases, and substantially increased RNA half-life in the blood, cerebrospinal fluid, or local extracellular microenvironment.

### 2. References and Definitions

The patent and scientific literature referred to herein establishes knowledge that is available to those of skill in the art. The issued U.S. patents, allowed applications, published foreign applications, and references, that are cited herein are hereby incorporated by reference to the same extent as if each was specifically and individually indicated to be incorporated by reference.

As used herein, the term “RNA agent” means an unmodified ribonucleic acid (RNA), modified RNA, or nucleoside surrogate.

As used herein with respect to RNA agents, the term “modified” means a molecule that differs in chemical structure from a naturally-occurring ribonucleic acid by one or more of the following changes: addition, subtraction or substitution of one or more atoms in the phosphodiester backbone (e.g., replacement of a phosphodiester linkage with a phosphorothioate or peptide nucleic acid linkage); addition, subtraction or substitution of one or more atoms in the ribose unit (e.g., replacement of a ribose unit with a deoxyribose unit); and addition subtraction or substitution of one or more atoms in the nucleoside base unit (e.g., replacement of a base unit with a base analog, as described herein). While referred to as “modified RNA,” this term may include molecules which are not technically RNAs (e.g., because ribose has been replaced).

As used herein, the term “nucleoside surrogate” means a molecule which differs from a naturally-occurring ribonucleic acid in that the ribophosphate backbone is replaced with a non-ribophosphate construct that allows the bases to the presented in the correct spatial relationship such that hybridization is substantially similar to what is seen with a ribophosphate backbone (e.g., non-charged mimics of a ribophosphate backbone).

As used herein, the term “iRNA agent” means an RNA agent which can, or which can be cleaved into an RNA agent which can, down regulate the expression of a target gene, preferably an endogenous or pathogen target RNA. iRNA agents include antisense RNA, micro-RNA and siRNA. While not wishing to be bound by theory, an iRNA agent may act by one or more of a number of mechanisms, including post-transcriptional cleavage of target mRNA (sometimes referred to in the art as RNAi), or pre-transcriptional or pre-translational mechanisms. An iRNA agent can include a single strand or can include more than one strand (e.g., a double-stranded iRNA agent). If the iRNA agent is a single strand, it is particularly preferred that it include a 5′ modification which includes one or more phosphate groups or one or more analogs of a phosphate group.

As used herein, the term “amphipathic” means comprised of both hydrophilic and hydrophobic moieties, and is synonymous with “amphiphilic.” Amphipathic molecules are capable of forming micelles when dispersed in aqueous solutions at an appropriate temperature and concentration.

As used herein, the term “critical micelle concentration” and the abbreviation “CMC” mean the concentration of an amphipathic compound at which micelles begin to spontaneously form in an aqueous solution. A “low” critical micelle concentration is less than 10⁻⁵ M.

As used herein, the term “molecular backbone length” refers to the sum of the bond lengths between the atoms constituting the longest continuous chain of atoms in a molecule, irrespective of bond angles. Thus, merely as an example, the molecular backbone length of isobutane is the sum of the bond lengths of three C—C bonds and the two C—H bonds (i.e., 3×1.43 Å+2×1.09 Å). Because this calculation ignores both bond angles and the fact that molecules can assume secondary structures (e.g., globular structures), it over-estimates the physical length of the molecules in situ. Nonetheless, it is a useful approximation for comparing the relative sizes of siRNA agents and hydrophilic chains for purposes of the invention.

As used herein, the term “decrease” means to decrease by at least 5% from a reference amount, as determined by a method and sample size that achieves statistical significance (i.e., p<0.1).

As used herein, the term “increase” means to increase by at least 5% from a reference amount, as determined by a method and sample size that achieves statistical significance.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, . . . , 0.9, 0.99, 0.999, or any other real values≧0 and ≦2, if the variable is inherently continuous.

As used herein, unless specifically indicated otherwise, the word “or” is used in the inclusive sense of “and/or” and not the exclusive sense of “either/or.”

### 3. Micelles Including Amphipathic Conjugates of iRNA Agents

Micelles are aggregates of amphipathic molecules dispersed in an aqueous medium forming what is called a colloidal solution. Mixed micelles are micelles comprising more than one type of amphipathic molecule. Commonly, micelles are formed from fatty acids (e.g., sodium stearate) and phospholipids (e.g., phosphatidyl ethanolamine). In aqueous solution, a micelle forms with the hydrophilic “head” regions at the exterior surface in contact with the surrounding solvent, and the hydrophobic “tail” regions buried in the interior of the micelle, isolated from the solvent. Micelles are generally spherical in shape but, because they are fluid, are deformable when subjected to hydrodynamic forces. Other shapes, such as ellipsoids and cylinders are also possible, depending upon the molecular geometry of its constituent molecule(s) and conditions such as amphipathic molecule concentration(s), temperature, pH, and ionic strength.

The present invention depends, in part, upon the recognition that mixed micelles can be produced that include iRNA moieties as part of an amphipathic conjugate, and in which the other amphipathic molecules forming the micelle have radially extending hydrophilic moieties that form a hydrophilic layer, and such that the iRNA moieties are positioned in said mixed micelle with at least a portion of their surfaces within the hydrophilic layer. This positioning of the iRNA moieties within the hydrophilic layer physically shields them from other molecules, and thereby decreases degradation of the iRNA moieties by nucleases in the local microenvironment of the micelle.

In order for the iRNA moieties to be positioned within, or at least partially within, the hydrophilic layer, the hydrophilic moieties or head regions of the micelle-forming amphipathic molecules must extend radially outward a distance sufficient to form a hydrophilic layer which can at least partially surround or mask the iRNA moiety. Thus, simple, unmodified fatty acids (e.g., stearic acid, palmitic acid), which have only carboxyl groups as hydrophilic moieties, are not useful in the invention. Similarly, most simple, unmodified phospholipids (e.g., phosphatidyl ethanolamine (PE), phosphatidyl choline (PC), phosphatidyl serine (PS)), which have small hydrophilic moieties, are not useful in the invention. Rather, useful micelle-forming amphipathic molecules include at least one hydrophilic chain which extends radially from the center of said mixed micelle. These hydrophilic chains can align themselves radially to form a hydrophilic layer.

Examples of hydrophilic chains include hydrophilic polymers, including but not limited to the following: polyethylene glycol (PEG), polyethyleneimine (PEI), polyvinylpyrrolidone, polyacrylamide, polyvinyl alcohol, polyoxazolines, polymorpholines, chitosan, and water-soluble peptides. These hydrophilic polymers can be of varying lengths, determined by the number of repeating polymer units, and can be described by the number of repeating units (e.g., PEG having 44 ethylene glycol units), the molecular weight in grams/mole (e.g., PEG having a molecular weight of 2000 grams/mole), or by the length of the molecular backbone (e.g., PEG having a molecular backbone of 200 Å).

Using standard chemistries well known in the art, micelle-forming molecules with small head regions can be modified by the addition of hydrophilic chains to produce micelle-forming amphipathic molecules useful in the invention. For example, the head regions of fatty acids or phospholipids can be covalently modified by esterification or other covalent reactions with hydrophilic chains. Thus, for example, hydrophilic chains such as PEG can form esters with fatty acids such as stearic acid to form amphipathic molecules such as stearyl-PEG, or with phospholipids such as PE to form amphipathic molecules such as PE-PEG.

As noted above, in order to be useful in the invention, the hydrophilic chains must extend radially outward a sufficient distance to at least partially surround or mask the iRNA moiety. Therefore, the required or desired length of the hydrophilic chains is a function of the size of the iRNA agent. Thus, if an iRNA agent extends radially outward a greater distance from the micelle center, it is desirable to form the mixed micelle using a micelle-forming amphipathic molecule with hydrophilic chains which also extend outward a greater distance.

For both siRNAs and micro-RNAs, which comprise double-stranded, 21-23 by iRNA moieties, the molecular backbone length (measured along the ribophosphate backbone) is approximately 192-210 Å (i.e., 9.15 Å per nucleotide times 21-23 nucleotides). If such an iRNA moiety is conjugated to a hydrophobic moiety by one of its 5′ or 3′ termini, the opposite end of the iRNA moiety can be expected to project radially outward from the hydrophobic core of the mixed micelle by approximately 192-210 Å. If the iRNA moiety is attached to the hydrophobic moiety of the iRNA conjugate by a linker of any length, the iRNA moiety may project even further from the hydrophobic core of the mixed micelle. Therefore, for an iRNA moiety which is an siRNA or micro-RNA moiety, the hydrophilic layer must, at a minimum, be approximately 192-210 Å in molecular backbone length in order to surround or mask the iRNA moiety on all sides. If a linker causes the iRNA moiety to project even further, a commensurately longer hydrophilic chain must be used. However, because RNA agents are subject to digestion from their ends by exonucleases, it may be desirable for the hydrophilic layer to extend further, such that the ends of the hydrophilic chains extend beyond the iRNA moiety, and the ends of the iRNA moiety are fully masked or buried within the hydrophilic layer. Thus, for example, hydrophilic chains having molecular backbone lengths of 200-2,000 Å may be employed in the invention. On the other hand, shorter hydrophilic chains may be employed (e.g., 50-200 Å) if the iRNA moiety adopts a coiled configuration which shortens its effective length, if partially surrounding the iRNA moiety is adequate to decrease digestion by nucleases, or if only partial protection from nucleases is required.

Antisense RNAs, which typically comprise single-stranded, 13-30 nucleotide iRNA moieties, the molecular backbone length (measured along the ribophosphate backbone) is approximately 119-275 Å (i.e., 9.15 Å per nucleotide times 13-30 nucleotides). Therefore, for an iRNA moiety which is an antisense RNA moiety, depending upon the length of the particular antisense RNA, the hydrophilic layer must, at a minimum, be approximately 119-275 Å in molecular backbone length in order to surround or mask the iRNA moiety on all sides. As before, the presence of linkers between the iRNA moieties and the hydrophobic moieties of the iRNA conjugate may require longer chains in the hydrophilic layer, and longer chains (e.g., 300-2,000 Å) or shorter chains (e.g., 50-200 Å) may be desired for the reasons set forth above.

Thus, in one aspect, the invention provides pharmaceutical formulations of an mRNA agent including (1) a mixed micelle including (a) a plurality of amphipathic iRNA conjugates, each of which includes a hydrophilic moiety and a hydrophobic moiety, and each of which hydrophilic moieties includes an iRNA moiety of the mRNA agent; (b) a plurality of micelle-forming amphipathic molecules, each of which includes a hydrophilic moiety and a hydrophobic moiety, and each of which hydrophilic moieties includes at least one hydrophilic chain which extends radially from the center of the mixed micelle; and (2) a pharmaceutically acceptable carrier.

In some embodiments of the pharmaceutical formulation, the plurality of hydrophilic chains forms a hydrophilic layer extending radially from the center of said mixed micelle; and the amphipathic mRNA conjugates are positioned in the mixed micelle such that at least a portion of the surface of the iRNA moiety is within the hydrophilic layer.

In some embodiments of the pharmaceutical formulation, no portion of the surface of the iRNA moiety extends radially outward beyond the hydrophilic layer.

In another aspect, the invention provides methods of increasing the delivery of an iRNA agent to an intracellular target by formulating the iRNA agent in one of the pharmaceutical formulations described herein, and administering the pharmaceutical formulation extracellularly, such that delivery of the iRNA agent to the intracellular target is increased relative to delivery of the iRNA agent in the pharmaceutically acceptable carrier. In some embodiments, the average amount of iRNA delivered to the intracellular target is increased by at least 10%, in some embodiments it is increased 10%-50%, in some embodiments it is increased 50%-100%, and in some embodiments it is increased greater than 2×.

In another aspect, the invention provides methods of decreasing extracellular nuclease degradation of an iRNA agent by formulating the iRNA agent in one of the pharmaceutical formulations described herein, and administering the pharmaceutical formulation extracellularly, such that extracellular nuclease degradation of the iRNA agent in the pharmaceutical formulation is decreased relative to degradation of the iRNA agent in the pharmaceutically acceptable carrier. In some embodiments, the amount of iRNA degraded by nuclease within the first hour of administration is decreased by at least 10%, in some embodiments it is decreased 10%-50%, in some embodiments it is decreased 50%-100%, and in some embodiments it is decreased greater than 2×.

### 4. Hydrophobic and Hydrophilic Moieties

A broad variety of monomers may be used to build polymeric hydrophobic cores as well as polymeric hydrophlic chains for use in the invention. See, for example, Torchilin (2007), “Micellar nanocarriers: pharmaceutical perspectives,” Pharm Res. 24(1):1-16.

For example, and without limitation, propylene oxide, aspartic acid, β-benzoyl-L-aspartate, γ-benzyl-L-glutamate, caprolactone, D,L-lactic acid, spermine, and many others can be used to form hydrophobic moieties using standard chemistries. Some of these monomers form hydrophobic polymeric blocks that can be used to produce the hydrophobic core of a micelle, while other compounds (e.g., lysine, spermine) form hydrophilic polymeric blocks which can bind oppositely charged hydrophobic substances to form a hydrophobic electrostatic complex, and the complex can form the core of a micelle. Block copolymers of poly(ortho esters) and PEG form 40-70 nm micelles with a CMC of around 10⁻⁴ g/l and can be lyophilized. Micelle-forming ABC-type triblock copolymers composed of monomethoxy-PEG, poly(2-(dimethylamino)ethyl methacrylate) and poly(2-(diethylamino)ethyl methacrylate), with the last component forming a hydrophobic core, can also be used and allow for the slow release of poorly soluble compounds which can be incorporated into the core. Polylactone-PEG double and triple block copolymers can be used as micelle-forming polymers, as well as poly(2-ethyl-2-oxazoline-block-poly(epsilon-caprolactone), which forms 20 nm micelles. Chitosan grafted with hydrophobic groups, such as palmitoyl, can also be used to prepare pharmaceutical micelles, and is highly biocompatible. Other materials which can be to prepare pharmaceutical micelles include copolymers of PEG and macromolecules, such as scorpion-like polymers and some star-like and core-shell constructs.

Phospholipid residues can also be successfully used as hydrophobic core-forming moieties. The use of lipid moieties as hydrophobic blocks capping hydrophilic polymer chains (e.g., PEG) can provide additional advantages for particle stability when compared with conventional amphiphilic polymer micelles due to the existence of two extremely hydrophobic fatty acid acyls, which can contribute considerably to an increase in the hydrophobic interactions between the polymeric chains in the micelle's core. A variety of conjugates of lipids with water-soluble polymers are commercially available, or can be easily synthesized. Diacyl-lipid-PEG conjugates have been introduced into the area of controlled drug delivery as polymeric surface-modifiers for liposomes. However, the diacyl-lipid-PEG molecule itself represents a characteristic amphiphilic polymer with a bulky hydrophilic (PEG) portion and a very short but very hydrophobic diacyl-lipid moiety. Similar to other PEG-containing amphiphilic block-copolymers, diacyl-lipid-PEG conjugates form micelles in an aqueous environment. A series of PEG-phosphatidylethanolamine (PEG-PE) conjugates have been synthesized using PE and N-hydroxysuccinimide esters of methoxy-PEG succinates (molecular weight of 2 kDa, 5 kDa and 12 kDa). All versions of PEG-PE conjugates form micelles with the size of 7 to 35 nm. No dissociation into individual polymeric chains was found following the chromatography of the serially diluted samples of PEG (5 kDa)-PE up to a polymer concentration of approximately 1 μg/ml, which corresponds to a micromolar CMC value, which is at least 100-fold lower than those of conventional detergents. With the size of PEG blocks going above 15 kDa, the stability of PEG-PE micelles begins to decrease. Preparation of lipid-based micelles by a detergent or water-miscible solvent removal method results in formation of particles with very similar diameters. Usually, such micelles have a spherical shape and uniform size distribution. Another important issue is that PEG₂₀₀₀-PE and PEG₅₀₀₀-PE micelles retain the size characteristic for micelles even after 48 h incubation in the blood plasma, i.e. the integrity of PEG-PE micelles should not be immediately affected by components of biological fluids upon parenteral administration.

Amphiphilic PVP-lipid conjugates with PVP block size between 1,500 and 8,000 Da have also been prepared, which easily form micelles in an aqueous environment. CMC values and the size of micelles formed depend on the length of the PVP block and vary between 10⁻⁴ and 10⁻⁶ M and 5 and 20 nm, respectively. Micelles prepared from a similar lipidated polymer, polyvinyl alcohol substituted with oleic acid, can also be used.

In some embodiments, the amphipathic micelle-forming molecule is a PEG-PE co-polymer. Polyethylene glycol (PEG) refers to an oligomer or polymer of ethylene oxide. Phosphatidyl ethanolamine (PE) is frequently the main lipid component of microbial membranes. It is a neutral or Zwitterionic phospholipid (at least in the pH range 2 to 7). In animal tissues, phosphatidyl ethanolamine tends to exist in diacyl, alkylacyl and alkenylacyl forms. In general, animal phosphatidyl ethanolamine tend to contain higher proportions of arachidonic and docosahexaenoic acids than the other Zwitterionic phospholipid, phosphatidyl choline. These polyunsaturated components are concentrated in position sn-2 with saturated fatty acids most abundant in position sn-1.

As noted above, in some embodiments of the pharmaceutical formulation, the plurality of hydrophilic chains forms a hydrophilic layer extending radially from the center of said mixed micelle; and the amphipathic iRNA conjugates are positioned in the mixed micelle such that at least a portion of the surface of the iRNA moiety is within the hydrophilic layer. In other embodiments, no portion of the surface of the iRNA moiety extends radially outward beyond the hydrophilic layer.

In some embodiments of the pharmaceutical formulation, the plurality of hydrophilic chains have an average backbone length between 200 and 500 Å. In other embodiments, the plurality of hydrophilic chains have an average backbone length between 100-1,000 Å, and between 50-2,000 Å.

In some embodiments of the pharmaceutical formulation, the plurality of hydrophilic chains have an average molecular weight between 1,500 and 5,000. In other embodiments, the plurality of hydrophilic chains have an average molecular weight between 500 and 15,000, and between 1,000-10,000.

In some embodiments of any of the foregoing, the hydrophobic moieties of the micelle-forming amphipathic molecules are selected from radicals of a long-chain fatty acid, a phospholipid, a lipid, and a glycolipid.

In some embodiments of any of the foregoing, the hydrophobic moieties of the amphipathic iRNA conjugates are selected from radicals of a cholesterol, a long chain fatty acid, a lipid, a phospholipid, and a glycolipid.

In some of the foregoing embodiments, the fatty acid is selected from butanoic acid, hexanoic acid, octanoic acid, decanoic acid, dodecanoic acid, tetradecanoic acid, hexadecanoic acid, octadecanoic acid, eicosanoic acid, docosanoic acid, tetracosanoic acid and unsaturated congeners thereof.

In some of the foregoing embodiments, the phospholipid is selected from phosphatidyl ethanolamine (PE), phosphatidyl choline (PC), phosphatidylglycerol (PG), phosphatidyl inositol (PI), phosphatidyl serine (PS), phosphatidic acid (PA), and sphingomyelin.

In some of the foregoing embodiments, the glycolipid is selected from a galactolipid, a sulfolipid, a cerebroside, and a ganglioside.

In some of embodiments of any of the foregoing, the hydrophilic moieties of the micelle-forming amphipathic molecules are selected from radicals of a PEG, a PEI, a polyvinylpyrrolidone, a polyacrylamide, a polyvinyl alcohol, a polyoxazolines, a polymorpholines, a chitosan, and a water-soluble peptide.

In some of the foregoing embodiments, the hydrophilic moieties are PEG radicals having an average molecular weight between 1,500 and 5,000 g/mole. In other embodiments, the PEG radicals have an average molecular weight between 500 and 15,000, and between 1,000-10,000.

In some embodiments the siRNA is conjugated to a lipophilic moiety such as cholesterol, another lipid or phospholipid.

In some preferred embodiments the delivery system is a mixed micelle comprising PEG-PE and siRNA-lipid conjugate.

### 5. Double-Stranded iRNA Agents and Moieties

Double-stranded RNA (dsRNA), including siRNA and micro-RNA, directs the sequence-specific silencing of mRNA through a process known as RNA interference (RNAi). The process occurs in a wide variety of organisms, including mammals and other vertebrates.

It has been demonstrated that 21-23 nt fragments of dsRNA are sequence-specific mediators of RNA silencing, e.g., by causing RNA degradation. While not wishing to be bound by theory, it may be that a molecular signal, which may be merely the specific length of the fragments, present in these 21-23 nt fragments recruits cellular factors that mediate RNAi.

Complementarity, or degree of homology with the target strand, is most critical in the antisense strand. While perfect complementarity is often desired, particularly in the antisense strand, some embodiments can include, particularly in the antisense strand, one or more but preferably 6, 5, 4, 3, 2, or fewer mismatches (with respect to the target RNA). The mismatches, particularly in the antisense strand, are most tolerated in the terminal regions and if present are preferably in a terminal region or regions, e.g., within 6, 5, 4, or 3 nucleotides of the 5′ and/or 3′ terminus. The sense strand need only be sufficiently complementary with the antisense strand to maintain the over all double strand character of the molecule.

As discussed elsewhere herein, an iRNA agent will often be modified. Single stranded regions of an iRNA agent will often be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an iRNA agent, e.g., against exonucleases, or to favor the antisense RNA agent to enter into RISC are also favored. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Exemplary modifications include the following:

(a) backbone modifications, e.g., modification of a backbone P, including replacement of P with S, or P substituted with alkyl or allyl, e.g., Me, and dithioates (S—P═S); these modifications can be used to promote nuclease resistance;

(b) 2′-O alkyl, e.g., 2′-OMe, 3′-O alkyl, e.g., 3′-OMe (at terminal and/or internal positions); these modifications can be used to promote nuclease resistance or to enhance binding of the sense to the antisense strand, the 3′ modifications can be used at the 5′ end of the sense strand to avoid sense strand activation by RISC;

(c) 2′-5′ linkages (with 2′-H, 2′-OH and 2′-OMe and with P═O or P═S) these modifications can be used to promote nuclease resistance or to inhibit binding of the sense to the antisense strand, or can be used at the 5′ end of the sense strand to avoid sense strand activation by RISC;

(d) L sugars (e.g., L ribose, L-arabinose with 2′-H, 2′-OH and 2′-OMe); these modifications can be used to promote nuclease resistance or to inhibit binding of the sense to the antisense strand, or can be used at the 5′ end of the sense strand to avoid sense strand activation by RISC;

(e) modified sugars (e.g., locked nucleic acids (LNA's), hexose nucleic acids (HNA's) and cyclohexene nucleic acids (CeNA's)); these modifications can be used to promote nuclease resistance or to inhibit binding of the sense to the antisense strand, or can be used at the 5′ end of the sense strand to avoid sense strand activation by RISC;

(f) nucleobase modifications (e.g., C-5 modified pyrimidines, N-2 modified purines, N-7 modified purines, N-6 modified purines), these modifications can be used to promote nuclease resistance or to enhance binding of the sense to the antisense strand;

(g) cationic groups and Zwitterionic groups (preferably at a terminus), these modifications can be used to promote nuclease resistance;

(h) conjugate groups (preferably at terminal positions), e.g., naproxen, biotin, cholesterol, ibuprofen, folic acid, peptides, and carbohydrates; these modifications can be used to promote nuclease resistance or to target the molecule, or can be used at the 5′ end of the sense strand to avoid sense strand activation by RISC.

Multiple asymmetric modifications can be introduced into either or both of the sense and antisense sequence. A sequence can have at least 2, 4, 6, 8, or more modifications and all or substantially all of the monomers of a sequence can be modified

### 6. Ligand-Conjugates, Carriers and Targeting

In order to target the pharmaceutical compositions of the invention to specific cell types, to increase the half-life of the pharmaceutical compositions (e.g., in the general circulation, or in cerebrospinal or lymphatic spaces), to increase the bioavailability of the pharmaceutical compositions, and/or to increase the cell-penetration/uptake of the pharmaceutical compositions, the pharmaceutical compositions can further comprise a ligand, carrier or targeting moiety which is (a) bound to the iRNA agent, (b) bound to a separate hydrophobic moiety or amphipathic moiety and included in the mixed micelle, and/or (c) bound to the hydrophilic chains of the hydrophilic layer. In some preferred embodiments, the ligand, carrier or targeting moiety is bound to the hydrophilic chains of the hydrophilic layer, and preferably the ends of the hydrophilic chains, so that it is exposed to the solvent or extracellular microenvironment, and is accessible. For example, the distal tips of the hydrophilic chains may be activated by various known chemistries, including without limitation p-nitrophenyl (pNP) carbonyl groups which may be used with PEG and other soluble block-forming polymers.

Thus, an iRNA agent, amphipathic moiety and/or hydrophilic chain can optionally contain a ligand-conjugated monomer subunit. The carrier (also referred to in some embodiments as a “linker”) can be a cyclic or acyclic moiety and includes two “backbone attachment points” (e.g., hydroxyl groups) and a ligand. The ligand can be directly attached (e.g., conjugated) to the carrier or indirectly attached (e.g., conjugated) to the carrier by an intervening tether (e.g., an acyclic chain of one or more atoms; or a nucleobase, e.g., a naturally occurring nucleobase optionally having one or more chemical modifications, e.g., an unusual base; or a universal base). In one embodiment, the carrier is hydroxyprolinol.

When bound to the iRNA agent, a ligand-conjugated monomer subunit may be the 5′ or 3′ terminal subunit of the iRNA molecule. Alternatively, the ligand-conjugated monomer subunit may occupy an internal position. More than one ligand-conjugated monomer subunit may be present in an iRNA agent. Preferred positions for inclusion of a tethered ligand-conjugated monomer subunits, e.g., one in which a lipophilic moiety, e.g., cholesterol, is tethered to the carrier are at the 3′ terminus, the 5′ terminus, or an internal position of the sense strand.

In some embodiments, a ligand alters the distribution, targeting or lifetime of an iRNA agent into which it is incorporated. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand.

Ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics.

Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of such polymers include polyamino acid, such as polylysine (PLL), poly L-aspartic acid, poly L-glutamic acid, and styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, sperm idine, polyamine, pseudopeptide-polyamine, peptidomimetic polyamine, dendrimer polyamine, arginine, amidine, protamine, cationic moieties, e.g., cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyamino acids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic.

Other examples of ligands include dyes, intercalating agents (e.g., acridines), cross-linkers (e.g., psoralene, mitomycin C), porphyrins (TPPC4, texaphyrin, sapphyrin), polycyclic aromatic hydrocarbons (e.g., phenazine, dihydrophenazine), artificial endonucleases (e.g., EDTA), lipophilic molecules, e.g., cholesterol, cholic acid, adamantane acetic acid, 1-pyrene butyric acid, dihydrotestosterone, glycerol (e.g., esters and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octadecyl)glycerol), geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetra-azamacrocycles), dinitrophenyl, HRP, or AP.

For example, dyes can be conjugated to the pharmaceutical formulations of the invention to act as imaging agents. Such imaging agents are useful for detecting the uptake of the iRNA agents of the invention by targeted cells, and for studying the pharmacokinetics of the pharmaceutical formulations of the invention. Such imaging agents can be produced by, for example, conjugating a chelating group to the hydrophilic chains of the hydrophilic layer, and chelating a heavy metal (e.g., ¹¹¹In, ⁹⁹Tc, Gd, Mn, Fe) to the chelating group for gamma or NMR imaging. As noted herein, fluorescent dyes (e.g., Cy3) can also be conjugated to the pharmaceutical formulations. Thus, in another aspect, the invention provides methods and products for detecting or imaging the uptake, localization, and/or pharmacokinetics of the mixed micelles of the invention.

Ligands can be proteins, e.g., glycoproteins, or peptides, e.g., molecules having a specific affinity for a co-ligand, or antibodies, e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the iRNA agent into the cell by activating an inflammatory response, for example. Exemplary ligands that would have such an effect include tumor necrosis factor alpha (TNFalpha), interleukin-1 beta, or gamma interferon.

In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation or cell-penetration agent. For example, Torchilin (2008), Adv Drug Deliv Rev. 60(4-5):548-58, discloses Tat peptide-mediated intracellular delivery of pharmaceutical nanocarriers. In some embodiments, the agent is amphipathic. Exemplary agents include a peptides such as TAT, antennopedia, penetratin, or arginine oligomers (e.g., R8 or R9). If the agent is a peptide, it can be modified, such as a peptidylmimetic or an invertomer, or include non-peptide or pseudo-peptide linkages, or the use of D-amino acids. A helical agent can be an alpha-helical agent, which in some embodiments can have a lipophilic and a lipophobic phase.

Peptides that target markers enriched in proliferating cells can be used. For example, RGD containing peptides and peptidomimetics can target cancer cells, in particular cells that exhibit an Iᵥθ₃ integrin. Thus, one could use RGD peptides, cyclic peptides containing RGD, RGD peptides that include D-amino acids, as well as synthetic RGD mimics. In addition to RGD, one can use other moieties that target the Iᵥ-θ₃ integrin ligand. Generally, such ligands can be used to control proliferating cells and angiogenesis. Conjugates of this type include a pharmaceutical composition that targets PECAM-1, VEGF, or a cancer gene, e.g., a cancer gene described herein.

A targeting agent that incorporates a sugar, e.g., galactose and/or analogues thereof, can also be useful. These agents target, in particular, the parenchymal cells of the liver. For example, a targeting moiety can include more than one or preferably two or three galactose moieties, spaced about 15 Å from each other. The targeting moiety can alternatively be lactose (e.g., three lactose moieties), which is glucose coupled to a galactose. The targeting moiety can also be N-Acetyl-Galactosamine, N-Ac-Glucosamine. A mannose or mannose-6-phosphate targeting moiety can be used for macrophage targeting.

The ligand can be a peptide or peptidomimetic. A peptidomimetic (also referred to as an oligopeptidomimetic) is a molecule capable of folding into a defined three-dimensional structure similar to a natural peptide. The attachment of peptide and peptidomimetics to the pharmaceutical compositions of the invention can affect pharmacokinetic distribution of the composition, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide.

An RGD peptide moiety can be used to target a tumor cell, such as an endothelial tumor cell or a breast cancer tumor cell (Zitzmann et al. (2002), Cancer Res. 62:5139-43). An RGD peptide can facilitate targeting to tumors of a variety of other tissues, including the lung, kidney, spleen, or liver (Aoki et al. (2001), Cancer Gene Therapy 8:783-787). In some embodiments, the RGD peptide will facilitate targeting of the pharmaceutical composition to the kidney. The RGD peptide can be linear or cyclic, and can be modified, e.g., glycosylated or methylated to facilitate targeting to specific tissues. For example, a glycosylated RGD peptide can deliver a pharmaceutical composition to a tumor cell expressing α_{V}β₃ (Haubner et al. (2001), J. Nucl. Med. 42:326-336).

A “cell permeation peptide” is capable of permeating a cell, such as a microbial cell (e.g., a bacterial or fungal cell) or a mammalian cell (e.g., a human cell). A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al. (2003), Nucleic Acids Res. 31:2717-2724).

In some embodiments, the ligand can be a substituted amine, e.g., dimethylamino. In certain embodiments the substituted amine can be rendered cationic, e.g., by quaternization, e.g., protonation or alkylation. In certain embodiments, the substituted amine can be at the terminal position of a relatively hydrophobic chain, e.g., an alkylene chain.

In some embodiments, the ligand can be one of the following triterpenes:

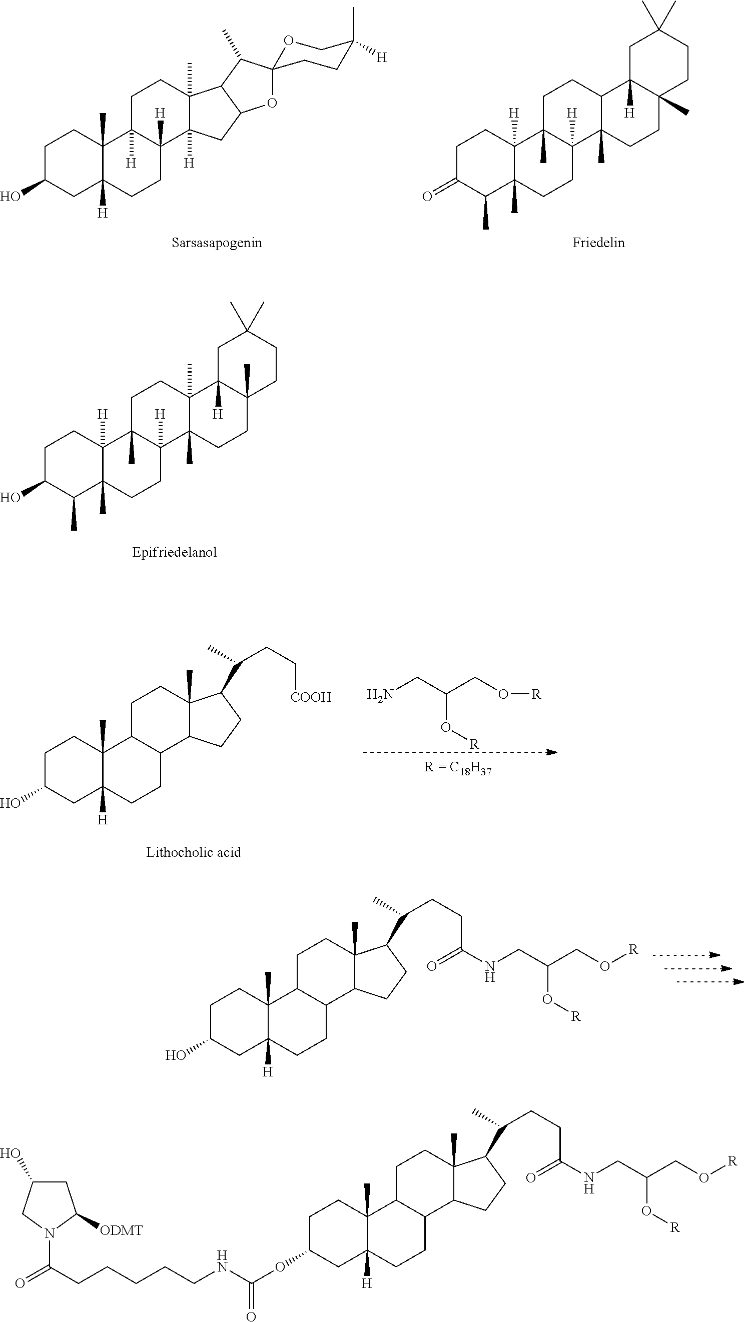

In some embodiments the ligand is cholesterol or another lipid or phospholipid.

iRNA agents include: molecules that are long enough to trigger the interferon response (which can be cleaved by the ribonuclease Dicer (Bernstein et al. (2001), Nature 409:363-366) and enter a RISC(RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed sRNA agents or shorter iRNA agents herein. “sRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60 but preferably less than 50, 40, or 30 nucleotide pairs. The sRNA agent, or a cleavage product thereof, can down regulate a target gene, e.g., by inducing RNAi with respect to a target RNA, preferably an endogenous or pathogen target RNA.

Each strand of a sRNA agent can be equal to or less than 30, 25, 24, 23, 22, 21, or 20 nucleotides in length. The strand is preferably at least 19 nucleotides in length. For example, each strand can be between 21 and 25 nucleotides in length. Preferred sRNA agents have a duplex region of 17, 18, 19, 29, 21, 22, 23, 24, or 25 nucleotide pairs, and one or more overhangs, preferably one or two 3′ overhangs, of 2-3 nucleotides.

In addition to homology to target RNA and the ability to down regulate a target gene, an iRNA agent can have one or more of the following properties:
(1) if single stranded it can have a 5′ modification which includes one or more phosphate groups or one or more analogs of a phosphate group;
(2) it can, despite modifications, even to a very large number, or all of the nucleosides, have an antisense strand that can present bases (or modified bases) in the proper three dimensional framework so as to be able to form correct base pairing and form a duplex structure with a homologous target RNA which is sufficient to allow down regulation of the target, e.g., by cleavage of the target RNA;
(3) it can, despite modifications, even to a very large number, or all of the nucleosides, still have “RNA-like” properties, i.e., it can possess the overall structural, chemical and physical properties of an RNA molecule, even though not exclusively, or even partly, of ribonucleotide-based content. For example, an iRNA agent can contain, e.g., a sense and/or an antisense strand in which all of the nucleotide sugars contain e.g., 2′ fluoro in place of 2′ hydroxyl. This deoxyribonucleotide-containing agent can still be expected to exhibit RNA-like properties. While not wishing to be bound by theory, the electronegative fluorine prefers an axial orientation when attached to the C2′ position of ribose. This spatial preference of fluorine can, in turn, force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. (Generally, it is preferred that a modified moiety at the 2′ sugar position will be able to enter into H-bonding which is more characteristic of the OH moiety of a ribonucleotide than the H moiety of a deoxyribonucleotide. For example, in some embodiments an iRNA agent can: exhibit a C_{3′}-endo pucker in all, or at least 50, 75, 80, 85, 90, or 95% of its sugars; exhibit a C_{3′}-endo pucker in a sufficient amount of its sugars that it can give rise to a the RNA-characteristic A-family-type helix; may have no more than 20, 10, 5, 4, 3, 2, or 1 sugar which is not a C_{3′}-endo pucker structure. These limitations may be preferable in the antisense strand;
(4) regardless of the nature of the modification, and even though the RNA agent can contain deoxynucleotides or modified deoxynucleotides, particularly in overhang or other single strand regions, it may be preferred that DNA molecules, or any molecule in which more than 50, 60, or 70% of the nucleotides in the molecule, or more than 50, 60, or 70% of the nucleotides in a duplexed region are deoxyribonucleotides, or modified deoxyribonucleotides which are deoxy at the 2′ position, are excluded from the definition of RNA agent.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents are preferably antisense with regard to the target molecule. In preferred embodiments single strand iRNA agents are 5′ phosphorylated or include a phosphoryl analog at the 5′ prime terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-). (These modifications can also be used with the antisense strand of a double stranded iRNA.)

A single strand iRNA agent should be sufficiently long that it can enter the RISC and participate in RISC mediated cleavage of a target mRNA. A single strand iRNA agent is at least 14, and more preferably at least 15, 20, 25, 29, 35, 40, or 50 nucleotides in length. It is preferably less than 200, 100, or 60 nucleotides in length.

Hairpin iRNA agents will have a duplex region equal to or at least 17, 18, 19, 29, 21, 22, 23, 24, or 25 nucleotide pairs. The duplex region will preferably be equal to or less than 200, 100, or 50, in length. Preferred ranges for the duplex region are 15-30, 17 to 23, 19 to 23, and 19 to 21 nucleotides pairs in length. The hairpin will preferably have a single strand overhang or terminal unpaired region, preferably the 3′, and preferably of the antisense side of the hairpin. Preferred overhangs are 2-3 nucleotides in length.

A “double stranded (ds) iRNA agent” as used herein, is an iRNA agent which includes more than one, and preferably two, strands in which interchain hybridization can form a region of duplex structure.

The antisense strand of a double stranded iRNA agent should be equal to or at least, 14, 15, 16 17, 18, 19, 25, 29, 40, or 60 nucleotides in length. It should be equal to or less than 200, 100, or 50, nucleotides in length. Preferred ranges are 17 to 25, 19 to 23, and 19 to 21 nucleotides in length.

The sense strand of a double stranded iRNA agent should be equal to or at least 14, 15, 16 17, 18, 19, 25, 29, 40, or 60 nucleotides in length. It should be equal to or less than 200, 100, or 50, nucleotides in length. Preferred ranges are 17 to 25, 19 to 23, and 19 to 21 nucleotides in length.

The double strand portion of a double stranded iRNA agent should be equal to or at least, 14, 15, 16 17, 18, 19, 20, 21, 22, 23, 24, 25, 29, 40, or 60 nucleotide pairs in length. It should be equal to or less than 200, 100, or 50, nucleotides pairs in length. Preferred ranges are 15-30, 17 to 23, 19 to 23, and 19 to 21 nucleotides pairs in length.

In many embodiments, the ds iRNA agent is sufficiently large that it can be cleaved by an endogenous molecule, e.g., by Dicer, to produce smaller ds iRNA agents, e.g., sRNAs agents

It is preferred that the sense and antisense strands be chosen such that the ds iRNA agent includes a single strand or unpaired region at one or both ends of the molecule. Thus, a ds iRNA agent contains sense and antisense strands, preferable paired to contain an overhang, e.g., one or two 5′ or 3′ overhangs but preferably a 3′ overhang of 2-3 nucleotides. Most embodiments will have a 3′ overhang. Preferred sRNA agents will have single-stranded overhangs, preferably 3′ overhangs, of 1 or preferably 2 or 3 nucleotides in length at each end. The overhangs can be the result of one strand being longer than the other, or the result of two strands of the same length being staggered. 5′ ends are preferably phosphorylated.

Preferred lengths for the duplexed region is between 15 and 30, most preferably 18, 19, 20, 21, 22, and 23 nucleotides in length, e.g., in the sRNA agent range discussed above. sRNA agents can resemble in length and structure the natural Dicer processed products from long dsRNAs. Embodiments in which the two strands of the sRNA agent are linked, e.g., covalently linked are also included. Hairpin, or other single strand structures which provide the required double stranded region, and preferably a 3′ overhang are also within the invention.

The isolated iRNA agents described herein, including ds iRNA agents and sRNA agents can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an sRNA agent of 21 to 23 nucleotides.

As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In one embodiment, an iRNA agent is “sufficiently complementary” to a target RNA, e.g., a target mRNA, such that the iRNA agent silences production of protein encoded by the target mRNA. In another embodiment, the iRNA agent is “exactly complementary” (excluding the RRMS containing subunit(s)) to a target RNA, e.g., the target RNA and the iRNA agent anneal, preferably to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the iRNA agent specifically discriminates a single-nucleotide difference. In this case, the iRNA agent only mediates RNAi if exact complementary is found in the region (e.g., within 7 nucleotides of) the single-nucleotide difference.

As used herein, the term “oligonucleotide” refers to a nucleic acid molecule (RNA or DNA) preferably of length less than 100, 200, 300, or 400 nucleotides.

RNA agents discussed herein include otherwise unmodified RNA as well as RNA which have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (1994), Nucleic Acids Res. 22: 2183-2196. Such rare or unusual RNAs, often termed modified RNAs (apparently because they are typically the result of a post transcriptionally modification) are within the term unmodified RNA, as used herein.

### REFERENCES

### General References

The oligoribonucleotides and oligoribonucleotides used in accordance with this invention may be with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotide-s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein.

Modification described in WO 00/44895, WO01/75164, or WO02/44321 can be used herein.

Phosphate Group References

The preparation of phosphonate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7, 651 and Crosstick et al. Tetrahedron Lett. 1989, 30, 4693.

Sugar Group References

Modifications to the 2′ modifications can be found in Verma et al. (1998), Annu. Rev. Biochem. 67:99-134 and all references therein. Specific modifications to the ribose can be found in the following references: 2′-fluoro (Kawasaki et al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

Replacement of the Phosphate Group References

Methylenemethylimino linked oligoribonucleosides, also identified herein as MMI linked oligoribonucleosides, methylenedimethylhydrazo linked oligoribonucleosides, also identified herein as MDH linked oligoribonucleosides, and methylenecarbonylamino linked oligonucleosides, also identified herein as amide-3 linked oligoribonucleosides, and methyleneaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 11972, 1991. Carbamate replacements are described in Stirchak, E. P. Nucleic Acids Res. 1989, 17, 6129.

Replacement of the Phosphate-Ribose Backbone References

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. No. 5,539,083.

Terminal Modification References

Terminal modifications are described in Manoharan, M. et al. Antisense and Nucleic Acid Drug Development 12, 103-128 (2002) and references therein.

Bases References

N-2 substituted purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references can be disclosed in the above section on base modifications.

### 7. Delivery Modules

The pharmaceutical compositions of the invention can be complexed to a delivery agent that features a modular complex. The complex can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane, e.g., an endosome membrane); and (c) a targeting group, e.g., a cell or tissue targeting agent, e.g., a lectin, glycoprotein, lipid or protein, e.g., an antibody, that binds to a specified cell type such as a kidney cell.

Thus, the pharmaceutical compositions described herein, can be linked, e.g., coupled or bound, to the modular complex. The pharmaceutical compositions can interact with the condensing agent of the complex, and the complex can be used to deliver an iRNA agent to a cell, e.g., in vitro or in vivo. For example, the complex can be used to deliver an iRNA agent to a subject in need thereof, e.g., to deliver an iRNA agent to a subject having a disorder, e.g., a disorder described herein, such as a disease or disorder of the kidney.

The fusogenic agent and the condensing agent can be different agents or the one and the same agent. For example, a polyamino chain, e.g., polyethyleneimine (PEI), can be the fusogenic and/or the condensing agent.

The delivery agent can be a modular complex. For example, the complex can include a carrier agent linked to one or more of (preferably two or more, more preferably all three of):

(a) a condensing agent (e.g., an agent capable of attracting, e.g., binding, a nucleic acid, e.g., through ionic interaction),

(b) a fusogenic agent (e.g., an agent capable of fusing and/or being transported through a cell membrane, e.g., an endosome membrane), and

(c) a targeting group, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine multivalent mannose, multivalent fucose, glycosylated polyamino acids, multivalent galactose, transferrin, bisphosphonate, polyglutamate, polyaspartate, a lipid, cholesterol, a steroid, bile acid, folate, vitamin B12, biotin, Neproxin, or an RGD peptide or RGD peptide mimetic.

7.1 Carrier Agents

The carrier agent of a modular complex described herein can be a substrate for attachment of one or more of: a condensing agent, a fusogenic agent, and a targeting group. The carrier agent would preferably lack an endogenous enzymatic activity. The agent would preferably be a biological molecule, preferably a macromolecule. Polymeric biological carriers are preferred. It would also be preferred that the carrier molecule be biodegradable.

The carrier agent can be a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or lipid. The carrier molecule can also be a recombinant or synthetic molecule, such as a synthetic polymer, e.g., a synthetic polyamino acid. Examples of synthetic polymers include polyamino acids, such as polylysine (PLL), poly L-aspartic acid, poly L-glutamic acid, and styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Other useful carrier molecules can be identified by routine methods.

A carrier agent can be characterized by one or more of: (a) is at least 1 Da in size; (b) has at least 5 charged groups, preferably between 5 and 5000 charged groups; (c) is present in the complex at a ratio of at least 1:1 carrier agent to fusogenic agent; (d) is present in the complex at a ratio of at least 1:1 carrier agent to condensing agent; (e) is present in the complex at a ratio of at least 1:1 carrier agent to targeting agent.

7.2 Fusogenic Agents

A fusogenic agent of a modular complex described herein can be an agent that is responsive to, e.g., changes charge depending on, the pH environment. Upon encountering the pH of an endosome, it can cause a physical change, e.g., a change in osmotic properties which disrupts or increases the permeability of the endosome membrane. In some embodiments, the fusogenic agent changes charge, e.g., becomes protonated, at pH lower than physiological range. For example, the fusogenic agent can become protonated at pH 4.5-6.5. The fusogenic agent can serve to release the iRNA agent into the cytoplasm of a cell after the complex is taken up, e.g., via endocytosis, by the cell, thereby increasing the cellular concentration of the iRNA agent in the cell.

In some embodiments, the fusogenic agent can have a moiety, e.g., an amino group, which, when exposed to a specified pH range, will undergo a change, e.g., in charge, e.g., protonation. The change in charge of the fusogenic agent can trigger a change, e.g., an osmotic change, in a vesicle, e.g., an endocytic vesicle, e.g., an endosome. For example, the fusogenic agent, upon being exposed to the pH environment of an endosome, will cause a solubility or osmotic change substantial enough to increase the porosity of (preferably, to rupture) the endosomal membrane.

The fusogenic agent can be a polymer, such as a polyamino chain, e.g., polyethyleneimine (PEI). The PEI can be linear, branched, synthetic or natural. The PEI can be, e.g., alkyl substituted PEI, or lipid substituted PEI.

In other embodiments, the fusogenic agent can be polyhistidine, polyimidazole, polypyridine, polypropyleneimine, mellitin, or a polyacetal substance, e.g., a cationic polyacetal. In some embodiments, the fusogenic agent can have an alpha helical structure. The fusogenic agent can be a membrane disruptive agent, e.g., mellittin.

A fusogenic agent can have one or more of the following characteristics: (a) is at least 1 Da in size; (b) has at least 10 charged groups, preferably between 10 and 5000 charged groups, more preferably between 50 and 1000 charged groups; (c) is present in the complex at a ratio of at least 1:1 fusogenic agent to carrier agent; (d) is present in the complex at a ratio of at least 1:1 fusogenic agent to condensing agent; (e) is present in the complex at a ratio of at least 1:1 fusogenic agent to targeting agent.

Other suitable fusogenic agents can be tested and identified by a skilled artisan. The ability of a compound to respond to, e.g., change charge depending on, the pH environment can be tested by routine methods, e.g., in a cellular assay. For example, a test compound is combined or contacted with a cell, and the cell is allowed to take up the test compound, e.g., by endocytosis. An endosome preparation can then be made from the contacted cells and the endosome preparation compared to an endosome preparation from control cells. A change, e.g., a decrease, in the endosome fraction from the contacted cell vs. the control cell indicates that the test compound can function as a fusogenic agent. Alternatively, the contacted cell and control cell can be evaluated, e.g., by microscopy, e.g., by light or electron microscopy, to determine a difference in endosome population in the cells. The test compound can be labeled. In another type of assay, a modular complex described herein is constructed using one or more test or putative fusogenic agents. The modular complex can be constructed using a labeled nucleic acid instead of the iRNA. The ability of the fusogenic agent to respond to, e.g., change charge depending on, the pH environment, once the modular complex is taken up by the cell, can be evaluated, e.g., by preparation of an endosome preparation, or by microscopy techniques, as described above. A two-step assay can also be performed, wherein a first assay evaluates the ability of a test compound alone to respond to, e.g., change charge depending on, the pH environment; and a second assay evaluates the ability of a modular complex that includes the test compound to respond to, e.g., change charge depending on, the pH environment.

7.3 Condensing Agent

The condensing agent of a modular complex described herein can interact with (e.g., attracts, holds, or binds to) a pharmaceutical composition of the invention to (a) condense, e.g., reduce the size or charge of the pharmaceutical composition and/or (b) protect the pharmaceutical composition, e.g., protect the iRNA agent against degradation. The condensing agent can include a moiety, e.g., a charged moiety, that can interact with a hydrophilic moiety, e.g., a hydrophilic chain or an iRNA agent, e.g., by ionic interactions. The condensing agent would preferably be a charged polymer, e.g., a polycationic chain. The condensing agent can be a polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

A condensing agent can have the following characteristics: (a) at least 1 Da in size; (b) has at least 2 charged groups, preferably between 2 and 100 charged groups; (c) is present in the complex at a ratio of at least 1:1 condensing agent to carrier agent; (d) is present in the complex at a ratio of at least 1:1 condensing agent to fusogenic agent; (e) is present in the complex at a ratio of at least 1:1 condensing agent to targeting agent.

Other suitable condensing agents can be tested and identified by a skilled artisan, e.g., by evaluating the ability of a test agent to interact with a hydrophilic moiety or an iRNA agent. The ability of a test agent to interact with a hydrophilic moiety or an iRNA agent, e.g., to condense or protect the iRNA agent, can be evaluated by routine techniques. In one assay, a test agent is contacted with a nucleic acid, and the size and/or charge of the contacted nucleic acid is evaluated by a technique suitable to detect changes in molecular mass and/or charge. Such techniques include non-denaturing gel electrophoresis, immunological methods, e.g., immunoprecipitation, gel filtration, ionic interaction chromatography, and the like. A test agent is identified as a condensing agent if it changes the mass and/or charge (preferably both) of the contacted hydrophilic moiety or nucleic acid, compared to a control. A two-step assay can also be performed, wherein a first assay evaluates the ability of a test compound alone to interact with, e.g., bind to, e.g., condense the charge and/or mass of, a hydrophilic moiety or nucleic cid; and a second assay evaluates the ability of a modular complex that includes the test compound to interact with, e.g., bind to, e.g., condense the charge and/or mass of, a hydrophilic moiety or nucleic acid.

Methods of Preparing Delivery Systems:

A delivery system described herein, e.g., a mixed micelle, can be prepared by co-suspending an amphipathic micelle-forming molecule and an iRNA-conjugate together, for example, in buffer. The amphipathic micelle-forming molecule and an iRNA-conjugate can be suspended together in various ratios, for example, in a molar ratio of 1:1. In some preferred embodiments, the micelle is prepared by suspending a 1:1 molar ratio of PEG-PE with a conjugated siRNA such as siRNA-Chol.

A preferred buffer includes a buffer having a pH of about 7, such as HBS buffer having a pH of about 7.4.

7.4 Second-Therapeutic Conjugates

A pharmaceutical composition of the invention can be coupled, e.g., covalently coupled, to a second therapeutic agent. For example, an iRNA agent used to treat a particular disorder can be coupled to a second therapeutic agent, e.g., an agent other than the mRNA agent. Alternatively, the second therapeutic agent can be coupled to a hydrophilic moiety in the hydrophilic layer of the mixed micelle, or to a hydrophobic moiety which is included in the hydrophobic core of the mixed micelle or, if it is hydrophobic, can be dissolved in the hydrophobic core of the micelle. The second therapeutic agent can be one which is directed to the treatment of the same disorder. For example, in the case of an iRNA used to treat a disorder characterized by unwanted cell proliferation, e.g., cancer, the iRNA agent or hydrophilic chains can be coupled to a second agent which has an anti-cancer effect. For example, the second therapeutic agent which stimulates the immune system, e.g., a CpG motif, or more generally an agent that activates a toll-like receptor and/or increases the production of gamma interferon.

The invention is further illustrated by the following examples, which should not be construed as further limiting.

### EXAMPLES

### PEG-PE Micelles for siRNA Delivery

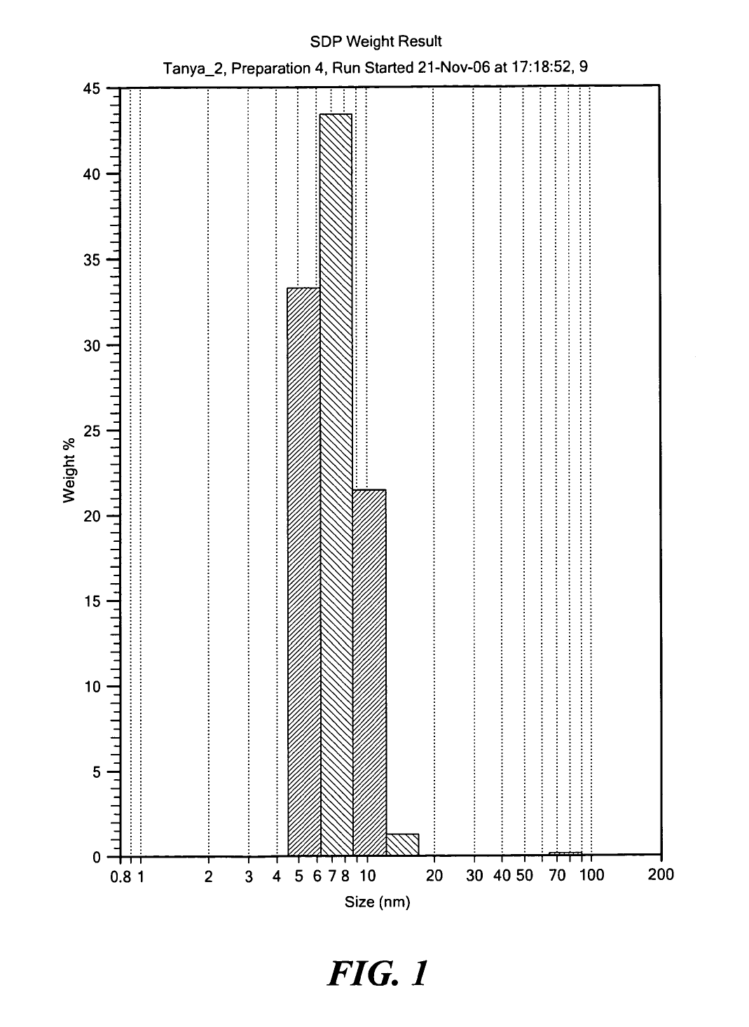

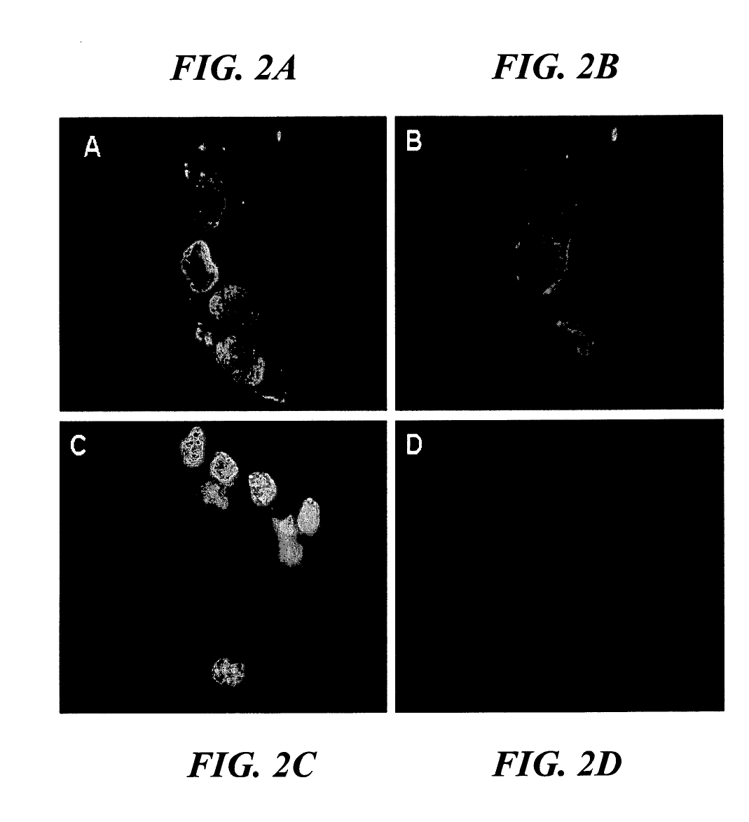

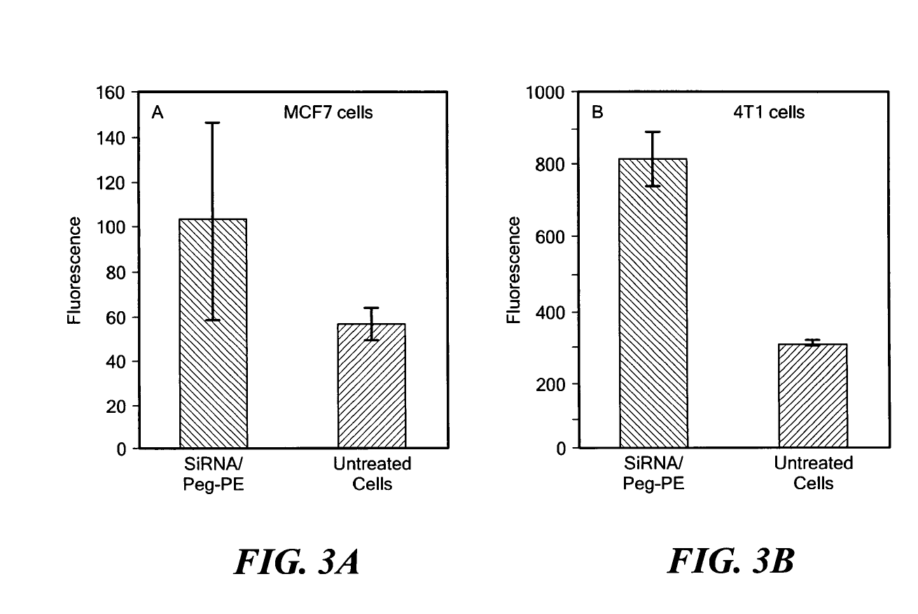

Effective intracellular delivery of siRNA is essential for its therapeutic activity. However, native siRNA doesn't always demonstrate the required high intracellular penetration. Various siRNA derivatives have been suggested to enhance its activity, such as siRNA-Chol. Polymeric micelles were prepared with high load of siRNA to enhance its uptake by the cells. To make such micelles, a mixed micelle system consisting of siRNA-Chol and PEG-PE conjugate was used, which demonstrated a very low CMC value and the ability to form stable mixed micelles with various amphiphiles. Cholesteryl-modified siRNA forms stable mixed micelles with PEG-PE, having a CMC value of approximately 6×10⁻⁶M (see FIG. 1), an average size of about 10 nm, and a narrow size distribution (see FIG. 2). The CMC values for the mixed siRNA-Chol/PEG-PE micelles remains the same when the siRNA moiety in siRNA-Chol is additionally modified with Cy3 dye. When incubated with MCF7 or 4T1 cancer cells, mixed siRNA-Chol/PEG-PE micelles with high siRNA load (approx. 50 mol %) and fluorescently labeled with Cy3 dye demonstrated effective cellular uptake within just 2 hours of incubation (see FIGS. 2, 3) and may serve as a convenient means to deliver siRNA into cells (FIGS. 2, 3).

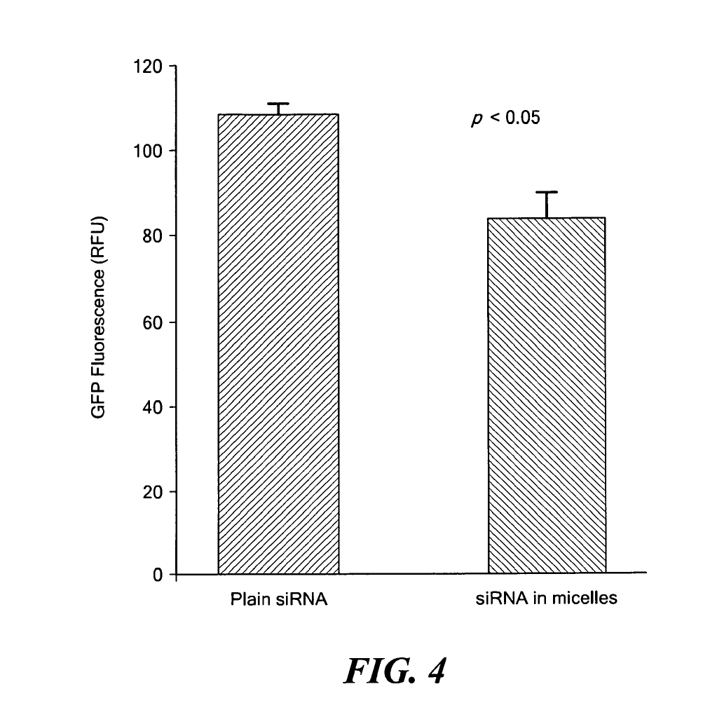

Preliminary experiments on the silencing property of micellar siRNA-Chol demonstrated that, in addition to intracellular uptake, siRNA-Chol/PEG-PE micelles were able to provoke a significant silencing of the target gene and decrease the GFP production in pGFP-transfected cells (FIG. 4). The first generation siRNA delivery system used in this experiment can be significantly optimized to make it highly effective.

Preparation of Mixed Micelles from PEG-PE and Chol-siRNA.

A commercial preparation of siRNA (silencing the GFP gene) can be used (Silencer®GFP (eGFP) siRNA cat#AM4626 from Ambion). An siRNA-Chol conjugate can be prepared by any standard chemistries, as described below. Mixed polymeric micelles can be prepared by co-suspending PEG-PE and Chol-siRNA (a small fraction of Chol-siRNA can be fluorescently labeled with Cy3 dye) in different molar ratios in HBS buffer, pH 7.4. The labeling of siRNA with Cy3 can be used for tracking purposes.

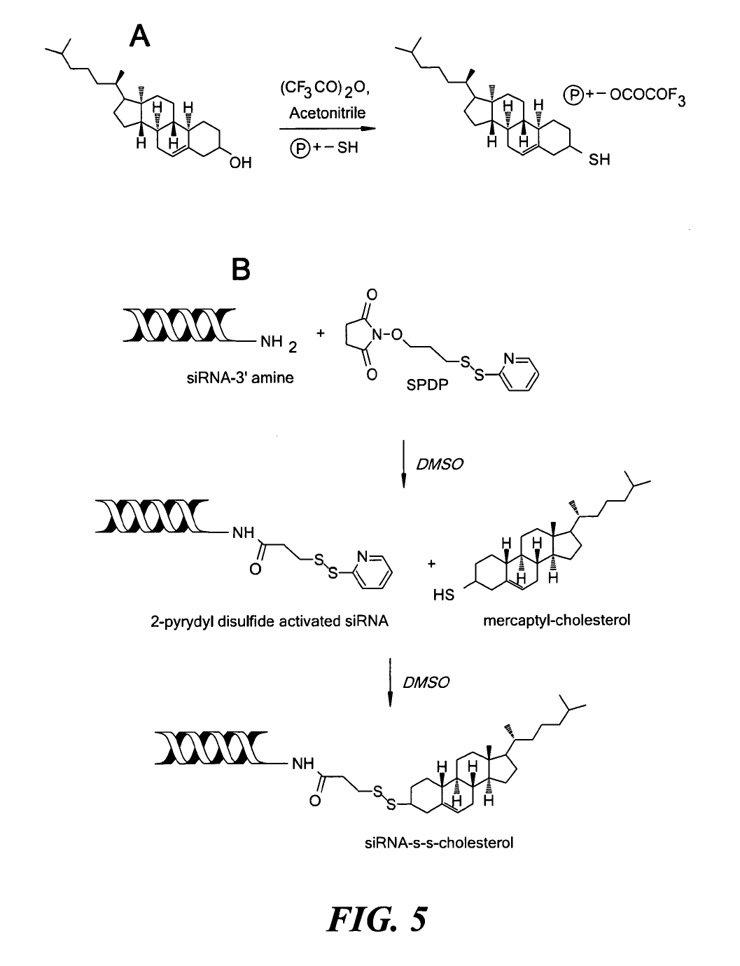

Synthesis of siRNA-Chol. Double stranded siRNA can be derivatized on both the 5′-end and 3′-end of the sense strand. Various functional groups bearing fluorescence (Chiu et al. (2002), Mol. Cell. 10:549-561; Harborth et al. (2003), Antisense Nucleic Acid Drug Dev. 13:83-105) or radioactivity (Braasch et al. (2004), Bioorg. Med. Chem. Lett. 14:1139-1143) as well as groups facilitating cellular uptake and transport through membranes (e.g., cholesterol, litocholic acid, lauryl acid or long alkyl chains) can be conjugated at this position (Soutschek et al. (2004), Nature 432:173-178; Lorenz et al. (2004), Bioorg. Med. Chem. Lett. 14:4975-4977). Delivery of cholesterol-modified siRNAs has been proven to be effective both in vitro (Lorenz et al. (2004), Bioorg. Med. Chem. Lett. 14:4975-4977) and in vivo (Soutschek et al. (2004), Nature 432:173-178), without addition of polycationic transfection reagents. Pendant groups such as puromycin (Chiu et al. (2002), Mol. Cell. 10:549-561), biotin (Chiu et al. (2002), Mol. Cell. 10:549-561), inverted nucleosides (Morrissey et al. (2005), Nat. Biotechnol. 23:1002-1007; Morrissey et al. (2005), Hepatology 41:1349-1356), allyl residue (Amarzguioui et al. (2003), Nucleic Acids Res 31:589-595) or aminoalkanes (Czauderna et al. (2003), Nucleic Acids Res 31:2705-2716) have been attached at the 3′-ends of siRNA molecules without any significant loss of silencing activity of resulting constructs. The 3′-amino-activated oligonucleotide can be conjugated to mercaptyl cholesterol via a disulfide linkage according to a published procedure (Bandgar et al. (2000), Chem. Lett. 29:1304-1305). For example, mercaptyl cholesterol can be prepared by direct synthesis of thiols from alcohols using trifluoroacetic anhydride and polymer supported hydrosulfide, under mild conditions. See, FIG. 5A. 3′ amino-siRNA can be conjugated to mercaptyl cholesterol using the heterofunctional spacer N-Succinimidyl-3-(2-pyridyldithio) propionate (SPDP) (Kim et al. (2006), J. Control. Release 116:123-129). See, FIG. 5B.

The characterization of siRNA-S—S-Chol can be performed by: (1) UV analysis for conjugation yield evaluation (λ=260 nm) (Lee et al. (2007), Biochem. Biophys. Res. Commun. 357:511-516); (2) Checking stability against the nuclease attack (Kim et al. (2006), J. Control. Release 116:123-129): using RNase protection assay, since it cleaves phosphodiester bond between any two ribonucleotides; (3) Evaluation of cleavability of the disulfide linkage in simulated intracellular conditions by incubation with a glutathione solution and subsequent analysis by electrophoresis; (4) Evaluation of serum stability by electrophoresis.

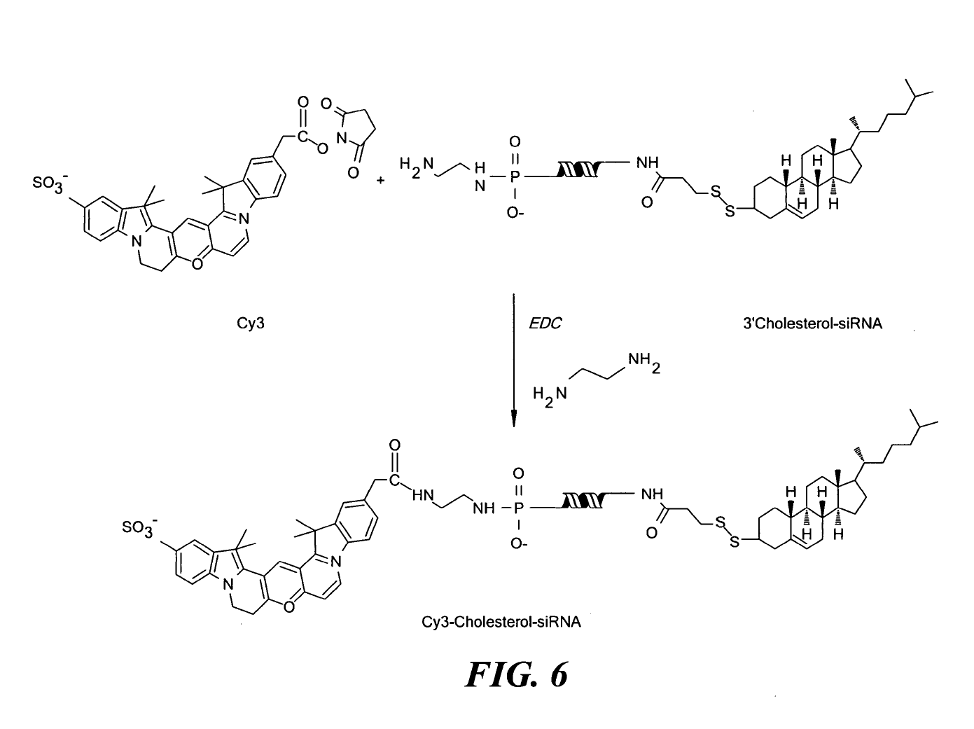

Labeling of siRNA-Chol with Cy3. When required, Cholesterol and Cy3 can be attached to the same siRNA moiety. Cholesterol-siRNA can be conjugated on the available phosphate group at a 5′ end using the water soluble carbodiimide EDC (1-ethyl-3-(3-dimethylamino-propyl) carbodiimide hydrochloride) producing a highly reactive phosphodiester intermediate; further reaction with ethylene diamine can provide a terminal amino reactive group. The activated carboxylic derivative Cy3B NHS Ester can be conjugated with the 5′-NH₂-3′-Cholesterol-siRNA to obtain Cy3 fluorescently labeled micelles. See FIG. 6.

1. A pharmaceutical formulation of an iRNA agent comprising:
(1) a mixed micelle comprising:
(a) a plurality of amphipathic iRNA conjugates, each of said conjugates comprising a hydrophilic moiety and a hydrophobic moiety, wherein each of said hydrophilic moieties comprises an iRNA moiety of said iRNA agent;
(b) a plurality of micelle-forming amphipathic molecules, each of said amphipathic molecules comprising a hydrophilic moiety and a hydrophobic moiety, wherein each of said hydrophilic moieties comprises at least one hydrophilic chain, which extends radially from the center of said mixed micelle; and
(2) a pharmaceutically acceptable carrier.

2. The pharmaceutical formulation of claim 1:
wherein said plurality of hydrophilic chains forms a hydrophilic layer extending radially from the center of said mixed micelle; and
wherein said amphipathic iRNA conjugates are positioned in said mixed micelle such that at least a portion of the surface of said iRNA moiety is within said hydrophilic layer.

3. The pharmaceutical formulation of claim 2:
wherein no portion of the surface of said iRNA moiety extends radially outward beyond said hydrophilic layer.

4. The pharmaceutical formulation of claim 2:
wherein said plurality of hydrophilic chains have an average backbone length of between 200 and 500 Å.

5. The pharmaceutical formulation of claim 2:
wherein said plurality of hydrophilic chains have an average molecular weight of between 1,500 and 5,000 g/mole.

6. The pharmaceutical formulation of claim 1, wherein said hydrophobic moieties of said micelle-forming amphipathic molecules are selected from the group consisting of radicals of a long-chain fatty acid, a phospholipid, a lipid, and a glycolipid.

7. The pharmaceutical formulation of claim 1, wherein said hydrophobic moieties of said amphipathic iRNA conjugates are selected from the group consisting of radicals of a cholesterol, a long chain fatty acid, a lipid, a phospholipid, and a glycolipid.

8. The pharmaceutical formulation of claim 6, wherein said fatty acid is selected from the group consisting of butanoic acid, hexanoic acid, octanoic acid, decanoic acid, dodecanoic acid, tetradecanoic acid, hexadecanoic acid, octadecanoic acid, eicosanoic acid, docosanoic acid, tetracosanoic acid and unsaturated congeners thereof.

9. The pharmaceutical formulation of claim 6, wherein said phospholipid is selected from the group consisting of phosphatidyl ethanolamine (PE), phosphatidyl choline (PC), phosphatidylglycerol (PG), phosphatidyl inositol (PI), phosphatidyl serine (PS), phosphatidic acid (PA), and sphingomyelin.

10. The pharmaceutical formulation of claim 6, wherein said glycolipid is selected from the group consisting of a galactolipid, a sulfolipid, a cerebroside, and a ganglioside.

11. The pharmaceutical formulation of claim 1, wherein said hydrophilic moieties of said micelle-forming amphipathic molecules are selected from the group consisting of radicals of a PEG, a PEI, a polyvinylpyrrolidone, a polyacrylamide, a polyvinyl alcohol, a polyoxazolines, a polymorpholines, a chitosan, and a water-soluble peptide.

12. The pharmaceutical formulation of claim 11 wherein said hydrophilic moieties are PEG radicals having an average molecular weight between 1,500 and 5,000 g/mole.

13. A method of increasing the delivery of an iRNA agent to an intracellular target comprising:
formulating said iRNA agent in a pharmaceutical formulation of claim 1; and
administering said pharmaceutical formulation extracellularly,
whereby delivery of said iRNA agent in said pharmaceutical formulation to said intracellular target is increased relative to delivery of said iRNA agent in said pharmaceutically acceptable carrier alone.

14. A method of decreasing extracellular nuclease degradation of an iRNA agent comprising:
formulating said iRNA agent in a pharmaceutical formulation of claim 1; and
administering said pharmaceutical formulation extracellularly,
whereby extracellular nuclease degradation of said iRNA agent in said pharmaceutical formulation is decreased relative to degradation of said iRNA agent in said pharmaceutically acceptable carrier alone.

15. The pharmaceutical formulation of claim 7, wherein said fatty acid is selected from the group consisting of butanoic acid, hexanoic acid, octanoic acid, decanoic acid, dodecanoic acid, tetradecanoic acid, hexadecanoic acid, octadecanoic acid, eicosanoic acid, docosanoic acid, tetracosanoic acid and unsaturated congeners thereof.

16. The pharmaceutical formulation of claim 7, wherein said phospholipid is selected from the group consisting of phosphatidyl ethanolamine (PE), phosphatidyl choline (PC), phosphatidylglycerol (PG), phosphatidyl inositol (PI), phosphatidyl serine (PS), phosphatidic acid (PA), and sphingomyelin.

17. The pharmaceutical formulation of claim 7, wherein said glycolipid is selected from the group consisting of a galactolipid, a sulfolipid, a cerebroside, and a ganglioside.

18. The pharmaceutical formulation of claim 1:
wherein, in each of said amphipathic iRNA conjugates, said hydrophilic moiety is conjugated to said hydrophobic moiety via a disulfide linkage.

19. A method of increasing the delivery of an iRNA agent to an intracellular target comprising:
formulating said iRNA agent in a pharmaceutical formulation of claims 18; and
administering said pharmaceutical formulation extracellularly,
whereby delivery of said iRNA agent in said pharmaceutical formulation to said intracellular target is increased relative to delivery of said iRNA agent in said pharmaceutically acceptable carrier alone.

20. A method of decreasing extracellular nuclease degradation of an iRNA agent comprising:
formulating said iRNA agent in a pharmaceutical formulation of claim 18; and
administering said pharmaceutical formulation extracellularly,
whereby extracellular nuclease degradation of said iRNA agent in said pharmaceutical formulation is decreased relative to degradation of said iRNA agent in said pharmaceutically acceptable carrier alone.
